(19) United States
(12) Patent Application Publication
(10) Pub. No.: US 2022/0018853 A1
(43) Pub. Date: Jan. 20, 2022

(54) USE OF GILZ AS A BIOMARKER IN SEPSIS

(71) Applicants: INSERM (INSTITUT NATIONAL DE LA SANTÉ ET DE LA RECHERCHE MÉDICALE), Paris (FR); UNIVERSITÉ PARIS-EST CRÉTEIL VAL DE MARNE, Creteil (FR); ASSISTANCE PUBLIQUE - HÔPITAUX DE PARIS (APHP), Paris (FR); HOPITAL FOCH, Suresnes France (FR); UNIVERSITÉ DE VERSAILLES SAINT-QUENTIN-EN-YVELINES, Versailles (FR)

(72) Inventors: Colas TCHERAKIAN, Suresnes France (FR); Djillali ANNANE, Garches France (FR); Yves LEVY, Creteil (FR); Véronique GODOT, Creteil - Cedex (FR)

(21) Appl. No.: 17/311,474

(22) PCT Filed: Dec. 6, 2019
(86) PCT No.: PCT/EP2019/083930

§ 371 Date: Jun. 7, 2021

(30) Foreign Application Priority Data
Dec. 7, 2018 (EP) 18306643.0

(51) Int. Cl. G01N 33/68; C12Q 1/6883

(52) U.S. Cl. CPC G01N 33/6893; C12Q 1/6883; C12Q 2600/118; G01N 2800/52; C12Q 2600/158; G01N 2800/26

(57) ABSTRACT

Septic shock is the leading cause of death in intensive care units. Previous studies have highlighted the immunosuppressive protein GILZ (glucocorticoid-induced leucine zipper) as a regulator of innate and adaptive immune responses. To go deeper in the understanding of GILZ protective role during sepsis, the inventors studied in vivo the consequences of a targeted overexpression of GILZ in monocytes and macrophages (M/M) in animal models of sepsis. In addition, they monitored the expression of GILZ in M/M of both patients with septic shock and septic mice. In particular, the inventors show that the overexpression of GILZ limited to M/M leads to an increase survival rate in mice with CLP-induced sepsis. These results provided new evidence for a central role of GILZ in M/M on the pathophysiology of septic shock, and pinpoint the fact that GILZ would be suitable for predicting survival time of patient suffering from sepsis. Moreover these results indicate that determining the level of GILZ expression level in monocytes/macrophages of patients suffering from sepsis is suitable for identifying those patients that will respond or not to treatment with a corticoid.

### FIELD OF THE INVENTION

The present invention is in the field of immunology.

### BACKGROUND OF THE INVENTION

Sepsis occurs when a site of infection is apparent and evidence shows deregulated body-response, resulting in life-threatening organs dysfunction. Corticosteroids include the natural steroid hormones produced by adrenocortical cells and a broad variety of synthetic analogues. Glucocorticoid effects include mainly regulation of carbohydrates, lipids and proteins metabolism, as well as regulation of inflammation. These molecular mechanisms of action of glucocorticoids were suggested to be appropriate for counteracting the uncontrolled inflammation that may characterize sepsis. Initially, researchers used high doses of corticosteroids, usually given as a single bolus, in an attempt to block potential bursts in pro-inflammatory cytokines. Recent systematic reviews and meta-analyses of trials of corticosteroids in sepsis found or did not find survival benefits from corticosteroids. Accordingly administration of corticosteroids in sepsis thus remains controversial and there is a need for biomarker to identify patients with septic shock who may best benefit from corticosteroids.

There is also a growing interest in understanding the effects of GC-induced proteins that may allow dissociation of GC anti-inflammatory effects from their adverse metabolic effects. Among the GC-induced proteins, GILZ (glucocorticoid-induced leucine zipper) is the focus of particular attention (7). GILZ, expressed in immune and non-immune cells including full range of actors involved in sepsis such as macrophages (8), neutrophils (9, 10), lymphocytes (11), dendritic cells (12, 13), mast cells (14) and endothelial cells (15), mediates GC immunosuppressive effects by inhibiting the activity of both pro-inflammatory transcription factors NF-κb, and AP-1(7). The impact of GILZ-driven changes varies from cell to cell. GILZ decreases the TNF secretion in monocytes (8), induces neutrophil apoptosis (10), favors T cell commitment to regulatory lineage (16), skews dendritic cell differentiation towards a tolerant state (12, 13, 17), and regulates vascular inflammation (15). The combination of all theses GILZ-mediated regulatory effects could explain the enhanced lifetime of transgenic mice with a global overexpression of GILZ during sepsis (18). Surprisingly, in mouse models of sepsis, the global overexpression of GILZ has no real impact on systemic inflammation (18). Also, global approaches of GILZ over-expression, i.e. in all cell types, may increase the chance of experiencing side effects, thus reducing the potential therapeutic effect of GILZ. There is much evidence demonstrating that GC immunosuppression is mediated by GILZ but there is very little specific information about GILZ involvement in GC-induced metabolic changes. GILZ is implicated in GC-mediated protein consumption in skeletal muscle cells (19) and its involvement in other metabolic abnormalities associated with GC has not yet been explored. It is submitted therefore that an overexpression of GILZ limited to relevant immune cells could be a good strategy to control excessive immune responses without altering the metabolic pathways. In this respect, the first challenge is to identify the target cell population, which may differ between immunopathologies.

### SUMMARY OF THE INVENTION

As defined by the claims, the present invention relates to the use of GILZ as a reliable biomarker in sepsis for predicting survival time and response to corticotherapy.

### DETAILED DESCRIPTION OF THE INVENTION

Septic shock, the leading cause of death in intensive care units, comes from an uncontrolled systemic inflammation triggered by infection and guided by macrophages. A recent clinical study supports the beneficial effects of an immunosuppressive corticotherapy during septic shock, and hence strengthens the focus on GC-induced proteins that may control the infectious inflammatory responses.

Previous studies have highlighted the immunosuppressive protein GILZ (glucocorticoid-induced leucine zipper) as a regulator of innate and adaptive immune responses. GILZ is a glucocorticoid-induced protein involved in the anti-inflammatory effects of glucocorticoids. In line with this, the generalized overexpression of GILZ, i.e. in all immune and non-immune cell types improves the outcome of septic shock in animal models but surprisingly with no real impact on the systemic inflammation.

To go deeper in the understanding of GILZ protective role during sepsis, the inventors studied in vivo the consequences of a targeted overexpression of GILZ in monocytes and macrophages (M/M) in animal models of sepsis. In addition, they monitored the expression of GILZ in M/M of both patients with septic shock and septic mice.

A significant down-regulation of GILZ was observed in patients' monocytes and in macrophages from septic mice compared to cells extracted from uninfected controls and was related to higher pro-inflammatory cytokine production. The overexpression of GILZ limited to M/M leads to an increase survival rate in mice with CLP-induced sepsis. Furthermore, the inventors determined that the up-regulation of GILZ in M/M reduced the systemic inflammation and the frequency of inflammatory monocytes while containing the bacterial spread during sepsis. They then showed in in vivo assays that peritoneal macrophages with an overexpression of GILZ have improved ingestion and killing capacities.

These results provided new evidence for a central role of GILZ in M/M on the pathophysiology of septic shock, hence a possible clue for modulation of inflammation and infection control in this severe disease.

Moreover these results indicate that determining the level of GILZ expression level in monocytes/macrophages of patients suffering from sepsis is suitable for identifying those patients that will respond or not to treatment with a corticoid.

Accordingly, the first object of the present invention relates to a method of predicting the survival time of a patient suffering from sepsis comprising the steps of:

i) providing a macrophage or monocyte sample from the patient,

ii) determining the expression level of GILZ in said sample,

iii) comparing the expression level determined at step ii) with a predetermined reference level and

iv) detecting differences between the expression level determined at step ii) and the predetermined reference value indicates that the patient will have a short or long survival time.

As used herein, the term “sepsis” has its general meaning in the art and represents a serious medical condition that is characterized by a whole-body inflammatory state. In addition to symptoms related to the provoking infection, sepsis is characterized by presence of acute inflammation present throughout the entire body, and is, therefore, frequently associated with fever and elevated white blood cell count (leukocytosis) or low white blood cell count and lower-than-average temperature, and vomiting. In particular, sepsis is defined as a deregulated immune response to infection, translating into life-threatening organs dysfunction, defined by a Sequential Organ Failure Assessment score of 2 more. Infection can be suspected or proven, or a clinical syndrome pathognomonic for infection. Septic shock is defined by infection and the need for vasopressors to maintain mean blood pressure >65 mmHg and arterial lactate levels >2 mmol/l.

In some embodiments, the subject suffers from SIRS. As used herein the term “SIRS” has its general meaning in the art and refers to systemic inflammatory response syndrome. IRS is characterized by hemodynamic compromise and resultant metabolic derangement. Outward physical symptoms of this response frequently include a high heart rate, high respiratory rate, elevated WBC count and elevated or lowered body temperature. Sepsis is differentiated from SIRS by the presence of a known pathogen. For example SIRS and a positive blood culture for a pathogen indicate the presence of sepsis.

In some embodiments, the septic patient suffers from acute respiratory distress syndrome. The term “acute respiratory distress syndrome” (abbreviated ARDS), as used herein, relates to a severe, life-threatening medical condition characterized by presence of a risk factor (e.g. pneumoniapancreatitis, etc.), bilateral pulmonary infiltrates, and oxygen impairment not fully explained by cardiac failure. More specifically, the term ARDS as used herein relates to acute respiratory distress syndrome as convened in 2011 in the Berlin definition (ARDS Definition Task Force et al. 2012 JAMA 307(23): 2526-2533).

As used herein, the expression “short survival time” indicates that the patient will have a survival time that will be lower than the median (or mean) observed in the general population of patients suffering from sepsis. When the patient will have a short survival time, it is meant that the patient will have a “poor prognosis”. Inversely, the expression “long survival time” indicates that the patient will have a survival time that will be higher than the median (or mean) observed in the general population of patients suffering from sepsis. When the patient will have a long survival time, it is meant that the patient will have a “good prognosis”.

A further object of the present invention relates to a method of determining whether a patient suffering from sepsis is eligible to treatment with a corticoid comprising the steps of:

i) providing a macrophage or monocyte sample from the patient before the treatment,

ii) determining the expression level of GILZ in said sample after an in vitro culture step in presence or absence of the selected corticosteroid,

iii) calculating the ratio between the expression levels determined at step ii)

iv) comparing the calculated ratio with a predetermined reference level and

v) detecting differences between the calculated ratio and the predetermined reference value indicates that the patient is eligible or not to the treatment.

As used herein, the term “corticosteroid”, used interchangeably with “corticoid” or “glucocorticoid”, refers to a class of therapeutic agents that bind cytosolic glucocorticoid receptor (GR) and are useful in treatment of inflammatory conditions. Corticosteroids include those that are naturally occurring, synthetic, or semi-synthetic in origin, and are typically characterized by the presence of a steroid nucleus of four fused rings, for example, as found in cholesterol, dihydroxycholesterol, stigmasterol, and lanosterol structures. Corticosteroid drugs include hydrocortisone (Cortisol), cortisone acetate, prednisone, prednisolone, methylprednisolone, deflazacort, betamethasone, triamcinolone, beclometasone, Paramethasone, fluticasone, fludrocortisone acetate, deoxycorticosterone acetate (DOCA), Fluprednisolone, fluticasone propionate, budesonide, beclomethasone dipropionate, flunisolide and triamcinolone acetonide. In some embodiments, the corticosteroid is dexamethasone.

As used herein the term “monocyte” has its general meaning in the art and is a large mononuclear phagocyte of the peripheral blood. Monocytes vary considerably, ranging in size from 10 to 30 μm in diameter. The nucleus to cytoplasm ratio ranges from 2:1 to 1:1. The nucleus is often band shaped (horseshoe), or reniform (kidney-shaped). It may fold over on top of itself, thus showing brainlike convolutions. No nucleoli are visible. The chromatin pattern is fine, and arranged in skein-like strands. The cytoplasm is abundant and appears blue gray with many fine azurophilic granules, giving a ground glass appearance in Giemsa staining. Vacuoles may be present. More preferably, the expression of specific surface antigens is used to determine whether a cell is a monocyte cell.

In some embodiments, the monocyte sample is a sample of blood monocytes.

As used herein the term “macrophage” has its general meaning in the art and refers to a cell exhibiting properties of phagocytosis. The morphology of macrophages varies among different tissues and between normal and pathologic states, and not all macrophages can be identified by morphology alone. However, most macrophages are large cells with a round or indented nucleus, a well-developed Golgi apparatus, abundant endocytotic vacuoles, lysosomes, and phagolysosomes, and a plasma membrane covered with ruffles or microvilli.

In some embodiments, the macrophage sample is sample of alveolar macrophages. As used herein, the term “alveolar macrophage” has its general meaning in the art and refers to a specific subset of macrophages that is present in the lung alveoli of a mammal. Methods for obtaining a population of alveolar macrophages from a mammal are conventional and typically include bronchial lavage.

Methods for isolating starting monocytes are well known in the art and include those described by Fluks A J. (1981); Hardin J A. et al. (1981); Harwood R. (1974); Elias J A et al. (1985); Brandslund I et al. (1982); Pertoft H et al. (1980); Nathanson S D et al. (1977); Loos H et al. (1976), Whal S M. et al. (1984). Macrophages and dendritic cells may be derived in vitro from monocytes by differentiation (Stanley et al., 1978, 1986; Gieseler R et al. 1998, Zhou et al. 1996; Cahpuis et al 1997, Brossart et al. 1998, Palucka et al 1998). In mice macrophages and DC may be obtained from spleen suspensions (Fukao, T., and Koyasu, S., 2000; Fukao, T., Matsuda, S., and Koyasu, S. 2000), from the peritoneal cavity (Mishell, B. B. and Shiigi, S. M. (1980) or most commonly from different bone marrow progenitors using various cytokine cocktails (Ardavin et al., 2001). One other standard method for isolating monocytes and macrophages consists in collecting a population of cells from a subject and using differential antibody binding, wherein cells of one or more certain differentiation stages are bound by antibodies to differentiation antigens. Fluorescence activated cell sorting (FACS) may be therefore used to separate the desired cells expressing selected differentiation antigens from the population of isolated cells. In some embodiments, magnetic beads may be used to isolate monocytes and macrophages cells from a cell population (MACS). For instance, magnetic beads labelled with monoclonal cell type specific antibodies may be used for the positive selection of human monocytes, from peripheral blood, or PBMCs, and of macrophages from pleural, peritoneal, or synovial fluids or from various tissues, such as spleen and lymph nodes. Other methods can include the isolation of monocytes by depletion of non-monocytes cells (negative selection). For instance non-monocytes cells may be magnetically labeled with a cocktail of monoclonal antibodies chosen antibodies directed against CD3, CD7, CD19, CD56, CD123 and CD235a. The main phenotypic markers of human monocyte cells include CD11b, CD11c, CD33 and CD115. Generally, human monocyte cells express CD9, CD11b, CD11c, CDw12, CD13, CD15, CDw17, CD31, CD32, CD33, CD35, CD36, CD38, CD43, CD49b, CD49e, CD49f, CD63, CD64, CD65s, CD68, CD84, CD85, CD86, CD87, CD89, CD91, CDw92, CD93, CD98, CD101, CD102, CD111, CD112, CD115, CD116, CD119, CDw121b, CDw123, CD127, CDw128, CDw131, CD147, CD155, CD156a, CD157, CD162, CD163, CD164, CD168, CD171, CD172a, CD180, CD206, CD131a1, CD213a2, CDw210, CD226, CD281, CD282, CD284, CD286 and optionally CD4, CD14, CD16, CD40, CD45RO, CD45RA, CD45RB, CD62L, CD74, CD142 and CD170, CD181, CD182, CD184, CD191, CD192, CD194, CD195, CD197, CX3CR1. Kits for isolation of monocytes, macrophages and dendritic cells are commercially available from Miltenyi Biotec (Auburn, Calif., USA), Stem Cells Technologies (Vancouver, Canada) or Dynal Bioech (Oslo, Norway).

Typically, the sample is contacted with the corticosteroid at step ii) for a time sufficient for inducing a possible increase in the expression level of GILZ. Typically, the sample is contacted for a time ranging from 30 min to 18 hrs. In some embodiments, the sample is contacted with the corticosteroid at step ii) for 30, 35, 40, 45, 50 or 55 min. In some embodiments, the sample is contacted with the corticosteroid at step ii) for 1, 2, 3, 4, 5, 6, 7, 8, 9, 10, 11, 12, 13, 14, 15, 16, 17, or 18 hours. The culture step is typically performed in any suitable container for performing in vitro culture and with any culture medium suitable for the culture of monocytes and macrophages.

As used herein, the term “GILZ” has its general meaning in the art and refers to Glucocorticoid-Induced Leucine Zipper protein. GILZ is also known as DIP; TSC22D3; DSIPI; or TSC-22R. An exemplary amino acid sequence is represented by SEQ ID NO:1.

>sp|Q99576|T22D3_HUMAN TSC22 domain family protein
3 OS = Homo sapiens OX = 9606 GN = TSC22D3 PE = 1
SV = 2
SEQ ID NO: 1
MNTEMYQTPMEVAVYQLHNFSISFFSSLLGGDVVSVKLDNSASGASVVAI
DNKIEQAMDLVKNHLMYAVREEVEILKEQIRELVEKNSQLERENTLLKTL
ASPEQLEKFQSCLSPEEPAPESPQVPEAPGGSAV

Methods for determining the expression level of a gene are well known in the art. The nucleic acid sample used for detecting the target sequence may be a DNA sample or an RNA sample. The latter may be preliminarily converted into cDNA before proceeding with said detection.

Conventional methods typically involve polymerase chain reaction (PCR). For instance, U.S. Pat. Nos. 4,683,202, 4,683,195, 4,800,159, and 4,965,188 disclose conventional PCR techniques. PCR typically employs two oligonucleotide primers that bind to a selected target nucleic acid sequence. Primers useful in the present invention include oligonucleotides capable of acting as a point of initiation of nucleic acid synthesis within the target nucleic acid sequence. A primer can be purified from a restriction digest by conventional methods, or it can be produced synthetically. If the template nucleic acid is double-stranded (e.g. DNA), it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min). If the double-stranded template nucleic acid is denatured by heat, the reaction mixture is allowed to cool to a temperature that promotes annealing of each primer to its target sequence on the target nucleic acid sequence. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min).

PCR involves use of a thermostable polymerase. The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished. Typically, the polymerase is a Taq polymerase (i.e. Thermus aquaticus polymerase).

The primers are combined with PCR reagents under reaction conditions that induce primer extension. Typically, chain extension reactions generally include 50 mM KCl, 10 mM Tris-HCl (pH 8.3), 15 mM MgCl2, 0.001% (w/v) gelatin, 0.5-1.0 μg denatured template DNA, 50 pmoles of each oligonucleotide primer, 2.5 U of Taq polymerase, and 10% DMSO. The reactions usually contain 150 to 320 μM each of dATP, dCTP, dTTP, dGTP, or one or more analogs thereof.

Quantitative PCR is typically carried out in a thermal cycler with the capacity to illuminate each sample with a beam of light of a specified wavelength and detect the fluorescence emitted by the excited fluorophore. The thermal cycler is also able to rapidly heat and chill samples, thereby taking advantage of the physicochemical properties of the nucleic acids and thermal polymerase.

In order to detect and measure the amount of amplicon (i.e. amplified target nucleic acid sequence) in the sample, a measurable signal has to be generated, which is proportional to the amount of amplified product. All current detection systems use fluorescent technologies. Some of them are non-specific techniques, and consequently only allow the detection of one target at a time. Alternatively, specific detection chemistries can distinguish between non-specific amplification and target amplification. These specific techniques can be used to multiplex the assay, i.e. detecting several different targets in the same assay. For example, SYBR® Green I probes, High Resolution Melting probes, TaqMan® probes, LNA® probes and Molecular Beacon probes can be suitable. TaqMan® probes are the most widely used type of probes. They were developed by Roche (Basel, Switzerland) and ABI (Foster City, USA) from an assay that originally used a radio-labelled probe (Holland et al. 1991), which consisted of a single-stranded probe sequence that was complementary to one of the strands of the amplicon. A fluorophore is attached to the 5′ end of the probe and a quencher to the 3′ end. The fluorophore is excited by the machine and passes its energy, via FRET (Fluorescence Resonance Energy Transfer) to the quencher. Traditionally, the FRET pair has been conjugated to FAM as the fluorophore and TAMRA as the quencher. In a well-designed probe, FAM does not fluoresce as it passes its energy onto TAMRA. As TAMRA fluorescence is detected at a different wavelength to FAM, the background level of FAM is low. The probe binds to the amplicon during each annealing step of the PCR. When the Taq polymerase extends from the primer which is bound to the amplicon, it displaces the 5′ end of the probe, which is then degraded by the 5′-3′ exonuclease activity of the Taq polymerase. Cleavage continues until the remaining probe melts off the amplicon. This process releases the fluorophore and quencher into solution, spatially separating them (compared to when they were held together by the probe). This leads to an irreversible increase in fluorescence from the FAM and a decrease in the TAMRA.

In some embodiments, the expression level of a gene can be determined at protein level. Typically, such methods comprise contacting the sample with at least one selective binding agent capable of selectively interacting with the protein of interest (i.e. GILZ). The selective binding agent may be polyclonal antibody or monoclonal antibody, an antibody fragment, synthetic antibodies, or other protein-specific agents such as nucleic acid or peptide aptamers. For the detection of the antibody that makes the presence of the marker detectable by microscopy or an automated analysis system, the antibodies may be tagged directly with detectable labels such as enzymes, chromogens or fluorescent probes or indirectly detected with a secondary antibody conjugated with detectable labels. The binding agents such as antibodies or aptamers may be labelled with a detectable molecule or substance, such as preferentially a fluorescent molecule, or a radioactive molecule or any others labels known in the art. As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof which is capable of exhibiting fluorescence in the detectable range. Labels of interest include both directly and indirectly detectable labels. Suitable labels for use in the methods described herein include any molecule that is indirectly or directly detectable by spectroscopic, photochemical, biochemical, immunochemical, electrical, optical, chemical, or other means. Labels of interest include, but are not limited to, fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Labels of interest also include fluorophores, such as indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Fluorescent labels can be detected using a photodetector (e.g., in a flow cytometer) to detect emitted light. Enzymatic labels are typically detected by providing the enzyme with a substrate and detecting the reaction product produced by the action of the enzyme on the substrate, colorimetric labels can be detected by simply visualizing the colored label, and antigenic labels can be detected by providing an antibody (or a binding fragment thereof) that specifically binds to the antigenic label. An antibody that specifically binds to an antigenic label can be directly or indirectly detectable. For example, the antibody can be conjugated to a label moiety (e.g., a fluorophore) that provides the signal (e.g., fluorescence); the antibody can be conjugated to an enzyme (e.g., peroxidase, alkaline phosphatase, etc.) that produces a detectable product (e.g., fluorescent product) when provided with an appropriate substrate (e.g., fluorescent-tyramide, FastRed, etc.); etc. The aforementioned assays may involve the binding of the binding agents (ie. antibodies or aptamers) to a solid support. The solid surface could be a microtitration plate coated with the binding partner. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, Calif.). According to the invention, methods of flow cytometry are preferred methods for measuring the level of the protein of interest (i.e. GILZ). Flow cytometry is a well-accepted tool in research that allows a user to rapidly analyze and sort components in a sample fluid. Flow cytometers use a carrier fluid (e.g., a sheath fluid) to pass the sample components, substantially one at a time, through a zone of illumination. Each sample component is illuminated by a light source, such as a laser, and light scattered by each sample component is detected and analyzed. The sample components can be separated based on their optical and other characteristics as they exit the zone of illumination. Said methods are well known in the art. For example, fluorescence activated cell sorting (FACS) may be therefore used and typically involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, data input devices, e.g., interface ports, a mouse, a keyboard, etc., fluid handling components, power sources, etc. Preferred methods typically involve the permeabilization of the cells (i.e. monocytes or macrophage) preliminary to flow cytometry. Any convenient means of permeabilizing cells may be used in practicing the methods.

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of ratios as calculated at step iii) in properly banked historical patient samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the ratio a group of reference (e.g. responder or non-responder), one can use algorithmic analysis for the statistic treatment of the calculated ratios in the samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, it is concluded that the patient will have a long survival time when the level determined at step ii) is higher than the predetermined reference value. Inversely, it is concluded that the patient will have a short survival time when the level determined at step ii) is lower than the predetermined reference value.

In some embodiments, it is concluded that the patient is eligible to the treatment when the ratio between the expression level determined in the presence of the corticosteroid and the expression level determined in the absence of the corticosteroid is higher than 1, 2, 3, 4, 5, 6, 7, 8, 9, or 10.

The method of the present invention is thus particularly suitable for predicting whether a patient suffering from sepsis will achieve a response with a corticosteroid. The term “predicting whether a patient will achieve a response”, as used herein refers to the determination of the likelihood that the patient will respond either favorably or unfavorably to the treatment. Especially, the term “prediction”, as used herein, relates to an individual assessment of any parameter that can be useful in determining the evolution of a patient. As will be understood by those skilled in the art, the prediction of the clinical response to the treatment, although preferred to be, need not be correct for 100% of the patients to be diagnosed or evaluated. The term, however, requires that a statistically significant portion of patients can be identified as having an increased probability of having a positive response. Whether a patient is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% at least 95%. The p-values are, preferably, 0.2, 0.1 or 0.05. As used herein, the term “response” or “responsiveness” refers to an improvement in at least one relevant clinical parameter as compared to an untreated patient diagnosed with the same pathology (e.g., the same type, stage, degree and/or classification of the pathology), or as compared to the clinical parameters of the same patient prior to treatment. In particular, the term “non responder” refers to a patient not experiencing an improvement in at least one of the clinical parameter and is diagnosed with the same condition as an untreated patient diagnosed with the same pathology (e.g., the same type, stage, degree and/or classification of the pathology), or experiencing the clinical parameters of the same patient prior to the treatment. Typically the response is associated with a decrease in the disease activity which can be determined by any conventional method well known in the art. In some embodiments, the response is survival.

A further object of the present invention relates to a method of treating a patient suffering from sepsis comprising i) determining whether the patient is eligible not to a treatment with a corticoid by performing the method of the present invention and ii) administering to the patient a therapeutically effective amount of a corticosteroid when it is concluded that the patient is eligible to said treatment.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

By a “therapeutically effective amount” of the corticosteroid as above described is meant a sufficient amount to provide a therapeutic effect. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, when it is concluded that the patient is not eligible to the treatment with the corticoid, the patient can then be managed according to the Surviving Sepsis Campaign guidelines (Dellinger R P, Levy M M, Rhodes A, Annane D, Gerlach H, Opal S M, et al. Surviving Sepsis Campaign: international guidelines for management of severe sepsis and septic shock, 2012. Intensive Care Med. (2013) 39:165-228.). In some embodiments, said treatment may consist in appropriate fluid therapy to resort preload, norepinephrine titrated to maintain mean blood pressure of 65 mmHg or more, oxygen supply, and broad spectrum antibiotics.

The invention will be further illustrated by the following figures and examples. However, these examples and figures should not be interpreted in any way as limiting the scope of the present invention.

### FIGURES

FIG. 1. GILZ is down regulated in peritoneal macrophages during sepsis

GILZ expression was quantified in peritoneal macrophages sorted from wild-type mice three hours after an i.p injection of LPS inducing endotoxemia (LPS from E. coli, 100 μg/mouse). Control mice received an i.p injection of PBS. (A) Gating strategy of LPM (CD45⁺CD11b^{high}F4/80^{high}) and SPM (CD45⁺CD11b^{int}F4/80⁺). (B) Frequency of LPM and SPM in the peritoneal cavity of mice after the PBS (white bars) or LPS injection (gray bars) (n=5). (C) GILZ, TNF and IL6 mRNA expression was quantified by qRT-PCR in the LPM and SPM sorted 3 hours after the injection of LPS (3H) or the PBS injection (PBS) (n=5). (D) Sorted mouse LPM (n=5) and (E) BM-DM (n=4) were stimulated in vitro with 100 ng/mL of LPS for indicated time or left unstimulated (Med) and then tested for the expression of GILZ, IL6 and TNF mRNA by qRT-PCR. In all qRT-PCR experiments, mRNA were normalized over β-actin expression. Results are expressed as mean±SEM. Two-tailed Mann-Whitney test or two-way ANOVA followed by Bonferroni post-hoc test was used to compare groups.*p<0.05, **p<0.01, ***p<0.005.

FIG. 2. GILZ is downregulated in LPS-exposed monocytes from healthy donors and in monocytes purified from septic shock patients

Levels of GILZ mRNA (A) as well as TNF secretion (A) in CD14+ monocytes isolated from healthy control donors and stimulated two hours with LPS from E. coli (100 ng/mL). Monocytes not exposed to LPS (Med or NS) served as control (n=6). (B) Correlation plot between GILZ mRNA and GILZ protein expression in human monocytes. (C) GILZ expression in monocytes purified from patients with septic shock or healthy donors (n=6 per group). Results are expressed as mean±SEM. Two-tailed Wilcoxon paired test (A), Spearman correlation test (B) or two-tailed Mann-Whitney test (C) were used to compare groups. *p<0.05, **p<0.01, ***p<0.005.

FIG. 3. Direct and negative link between GILZ expression and LPS-induced cytokine secretions by human and murine M/M

Human monocytes were transfected with a plasmid encoding GILZ (pGILZ) or the control plasmid (pCtrl) and tested for their expression of GILZ mRNA (A) (n=6). Their secretion of TNF was measured by ELISA in the culture supernatants two hours after LPS-exposure (B) (n=6). LPM were isolated from GILZ^{high} or control mice (wt) and tested for the expression of GILZ by qRT-PCR (C) (3 mice per group, measurements done in triplicate, one representative experiment out of 3 is shown) (D) Expression of GILZ mRNA in alveolar macrophages (CD45⁺CD11c⁺F4/80⁺) isolated from GILZ^{high} or control mice (wt) (3 mice per group, measurements done in triplicate, one representative experiment out of 3 is shown). Levels of (E) GILZ mRNA, (F) TNF, IL10, CCL2 and IL6 mRNA in LPM sorted from GILZ^{high} (black bars) or control mice (white bars) and stimulated four hours with LPS (100 ng/mL). mRNA have been quantified by qRT-PCR and normalized over β-actin expression (3 mice per group, measurements done in triplicate, one representative experiment out of 3 is shown). (G) LPM were harvested from GILZ^{high} (black bars) or control (white bars) mice four hours after an i.p. injection of PBS or LPS (100 μg/mouse) and tested by flow cytometry for their expression of TLR2 and TLR4 (n=4).

Results are expressed as mean±SEM. Two-tailed Mann-Whitney test or two-way ANOVA followed by Bonferonni post-hoc test were used to compare groups. *p<0.05, **p<0.01, ***p<0.005, ^{ns}p>0.05.

FIG. 4. Alteration of systemic endotoxin-related inflammatory responses in GILZ^{high} mice

GILZ^{high} (black bar) and control mice (wt, white bar) were injected i.p. with LPS. (A-B) The levels of cytokines and chemokines were assessed in the plasma six hours after the LPS injection using a Luminex assay (n=4). Cytokines were not detectable in the plasma of PBS-injected mice. (C) Frequency of neutrophils and (D) Ly6C⁺ or Ly6C⁻ monocyte subsets (CD45⁺CD115⁺) in LPS-injected GILZ^{high} and control mice (wt) for indicated time (n=4 per group). (E-F) Survival under severe-grade (E) and mild-grade (F) polymicrobial septic shock induced by cecal ligation and puncture (n=12 per group and per CLP model). (G) Lactate concentrations and (H) bacteremia assessed in the blood of mice 18 hours after the induction of a mild-grade CLP (n=12).

Results are expressed as mean±SEM. Two-tailed Mann-Whitney test or two-way

ANOVA followed by Bonferroni post-hoc test or log-Rank test was applied for group comparisons. *p<0.05, **p<0.01, ^{ns}p>0.05.

FIG. 5. Increased E. coli phagocytosis by GILZ^{high} macrophages

In vivo phagocytosis assay where pHodro-green conjugated E. coli were injected i.p. in GILZ^{high} or control mice (wt). Thirty minutes and two hours after the injection, peritoneal macrophages were recovered and further stained to identify viable SPM and LPM. (A-B) Viable LPM and SPM (C-D) were analyzed by flow cytometry for the detection of pHodro-green signal (n=6 per group).

The results are expressed as mean±SEM. Two-tailed Mann-Whitney test was used to compare groups. *p<0.05, **p<0.01.

### EXAMPLE

Material and Methods

Mice

Mice aged between 8 and 14 weeks were used. The homozygous GILZ^{high} transgenic mice carry a transgene encoding mouse GILZ under the direction of the CD68 promoter (20). Congenic control mice used as control had been obtained by crossing the GILZ^{high} heterozygous mice. Experiments were approved by the local Ethics Committee for Animals (CEEA-16, Cometh, Maison-Alfort, France, agreement number 028-245, project number 02858.01) and complied with French and European guidelines for the use of laboratory animals.

Septic Shock Patients

Patients admitted to the intensive care unit at Raymond-Poincaré Hospital (Garches, France), were included if they had: 1) at least one proven site of infection; 2) multiple organ failure as defined by a Sepsis-related Organ Failure Assessment score (SOFA score) above six for >6 consecutive hours (21); and 3) need for norepinephrine infusion to stabilize mean arterial blood pressure over 65 mmHg. Four milliliters of peripheral venous blood were collected in EDTA-tubes for purification of monocytes and monitoring of GILZ expression by qRT-PCR. Plasma was clarified by two successive centrifugations at 600 g and 10 000 g for 10 minutes and stored at −80° C. until analysis. Protocols were approved by the Comité de Protection des Personnes de Saint-Germain-en-Laye (France). Healthy gender- and age-matched donors were used as controls. All participants gave signed informed consent.

Cell Purification

CD14+ blood monocytes were purified according to the manufacturer's instructions using CD14+ microbeads (Miltenyi Biotec). Untouched whole blood monocytes were magnetically isolated using the Human monocytes isolation kit without CD16 depletion from StemCell completed with a tetrameric complex against CD94, CD61, KIR3DL1 (StemCell) with an average purity of 93.77%+/−3.1%.

Peritoneal murine macrophages were sorted with a FACS Aria using Diva software (Becton Dickinson) as previously reported (22). For in vitro experiments, macrophages were left overnight in complete medium before stimulation. For mRNA quantification, cells were directly lysed in 3004, of lysis buffer (RLT+, Qiagen).

Cell Culture and Stimulation

Human monocytes were cultured in RPMI 1640 medium (Gibco) plus 10% human AB serum (PAA, GE healthcare), 25 mM HEPES (Gibco) and 1% penicillin/streptomycin (Gibco). Monocyte transfection was performed with a plasmid encoding GILZ or the empty plasmid as we described in a previous report (8).

Mouse macrophages were cultured in RPMI 1640 medium plus 10% fetal calf serum (GE Healthcare), HEPES 25 mM (Gibco), 1% non-essential amino acids (Gibco), and 1% penicillin/streptomycin (Gibco).

Bone marrow derived macrophages (BM-DM) generated according to the protocol described in our previous study (23) were washed twice in PBS 1× and seeded overnight at 1×10⁶ cells/mL in complete medium before their use in in vitro stimulation assays where cells were stimulated with 100 ng/mL of Escherichia coli LPS (055:B5, Sigma-Aldrich) for the indicated time.

Endotoxemia and Septic Shock Assay

A Sub-lethal endotoxin model was obtained by i.p. injection of LPS (100 μg/mouse, E. Coli 055:B5, ENZO Lifescience). Polymicrobial sepsis was induced by cecal ligation and puncture (CLP) as we previously described (23). Severe-grade and mild-grade CLP were obtained by using two different lengths of cecal ligation (24). In the severe-grade CLP, two punctures were made in the cecum with a 21-gauge needle on animals having a ligation area of 1.5 cm. In the mild-grade CLP, the two punctures were made on animals having a ligation area of 1 cm. In both procedures, a small amount of cecal content was extruded from the perforation sites before replacing the cecum into the peritoneal cavity.

Blood samples were collected in tubes containing EDTA at termination by cardiac puncture or from the retromandibular vein of anesthetized mice according to the design of the experiment. The clarified plasma samples were stored at −80° C.

Flow Cytometry

After a blocking step using anti-CD16/CD32 antibodies, staining procedure was performed during 30 minutes at 4° C. in PBS 2% FCS. Anti-mouse F4/80 (BM8), CD11b (M1/70), CD45 (30-F11), Ly6C (HK1.4), Ly6G (RB6-8C5), NK1.1 (PK136) and anti-human CD14 (61D3), CD45 (2D1), CD16 (3G8) antibodies were purchased from eBioscience (San Diego, USA). Anti-mouse CD4 (4SM95), CD3 (17A2), CD8 (SK1), CD11c (N418), CD19 (HIB19), CD115 (AFS98), TLR4 (MTS510), TLR2 (1167) were purchased from Becton Dickinson. Acquisitions were performed on a LSRFortessa™ analyzer (Becton Dickinson). Data were analyzed using FlowJo software (FlowJo LLC).

Cytokine Quantification

Murine cytokines and chemokines were measured with 26-plex Luminex assay (eBioscience) on a bioplex 200 (Bio-Rad Laboratories) according to the manufacturer's instructions. Human cytokines were quantified by ELISA (Diaclone).

Determination of Blood Bacterial Load

Serial dilutions of blood collected by cardiac puncture were prepared with sterile PBS for plating on blood agar plates (Biomerieux). Plates were incubated at 37° C. overnight. Viable counts of bacteria were expressed as colony-forming units (CFU) per mL of blood.

In Vivo Phagocytosis Assays

One hundred micrograms of fluorescent BioParticles were administrated to mice through the peritoneum (i.p. injection). Peritoneal cells were harvested at two time points post-injection (30 min and 2 hours), washed, stained on ice and kept on ice in the living cell imaging solution before analysis by flow cytometry.

RNA Extraction and Quantitative RT-PCR

RNA extraction was performed using RNeasy mini or micro kit Plus (Qiagen) according to the manufacturer's instructions. cDNA was obtained by reverse transcription using a first strand cDNA synthesis Kit (Stratagene). Quantitative PCR reactions were performed using Brilliant II SYBR Green QPCR master Mix in a Mx3005P thermal cycler (Stratagene) according to the manufacturer's instructions. Relative expression of target genes was calculated and normalized to β-actin by the standard curve method. All primers used for qPCR are listed in the supplemental table 51.

Western-Blot Analysis

Western blotting was performed as previously described with the following antibodies: anti-GILZ (Santa-Cruz) and anti-GAPDH (eBiosciences) (17). Donkey anti-mouse and mouse anti-rabbit HRP conjugated antibodies were purchased from Lifetechnologies.

Statistics

Experiments with more than two groups or multiple comparisons were analyzed by two-way ANOVA followed by a Bonferroni post-hoc test. Experiments with two groups were analyzed by Mann-and-Whitney unpaired or Wilcoxon paired test depending on the experimental design. Log-rank tests were used in survival assays. Each test was considered statistically significant if p value was under 0.05 in two-tailed tests. All analyses were performed on Prism software (GraphPad, San Diego, USA).

Results:

GILZ is Downregulated in M/M During Sepsis

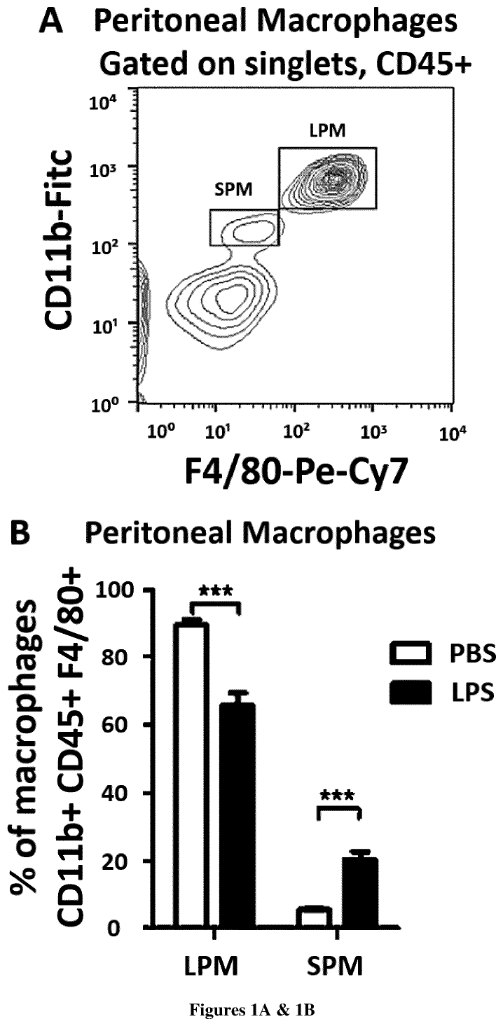

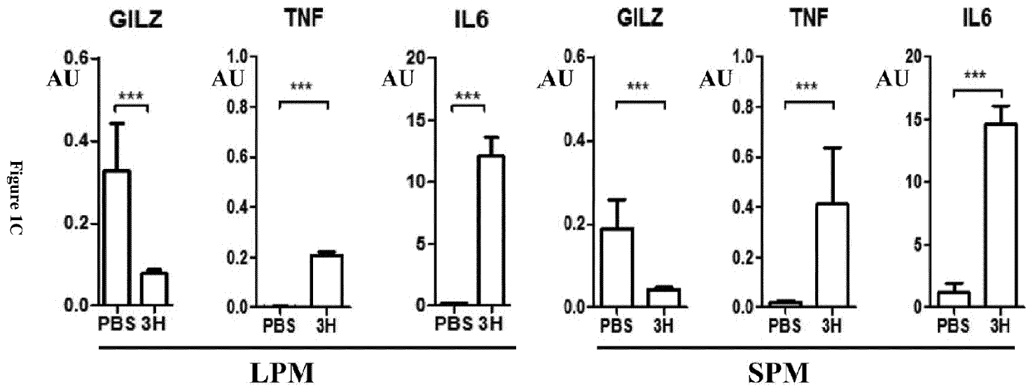

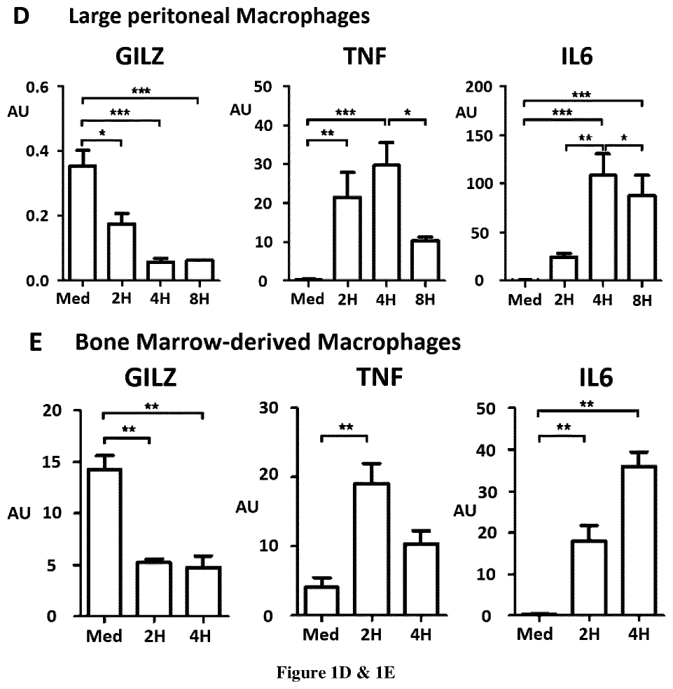

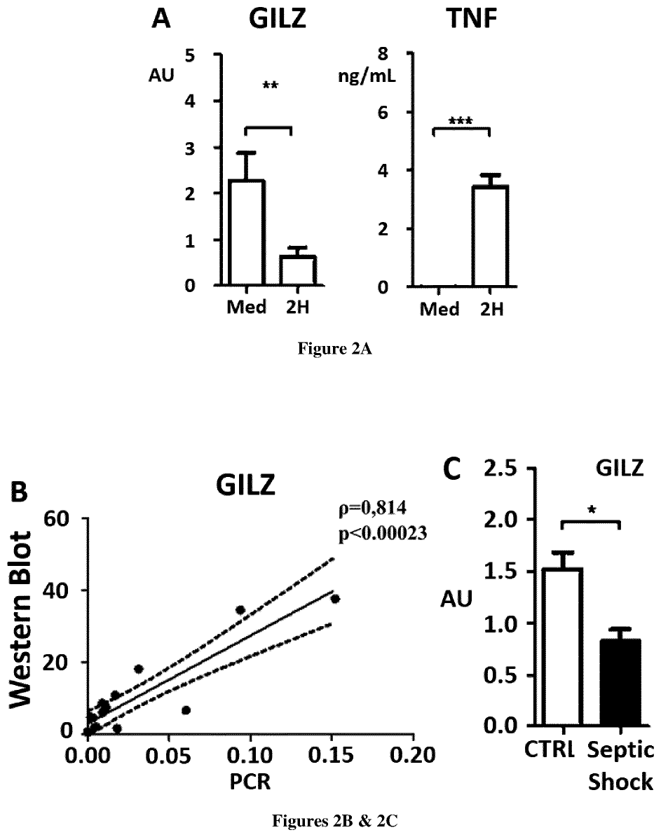

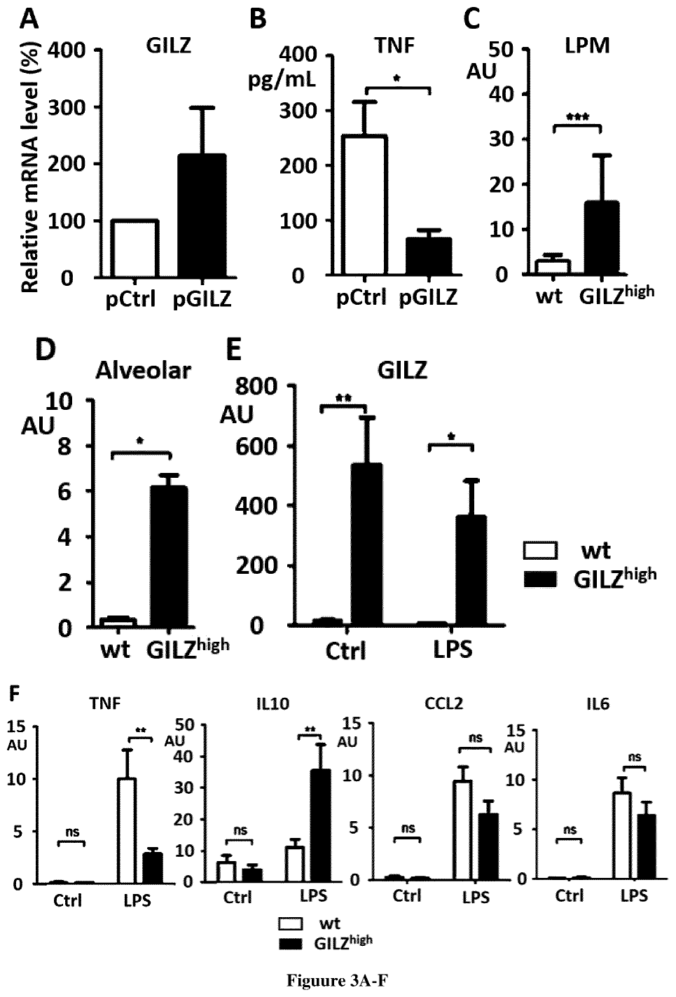

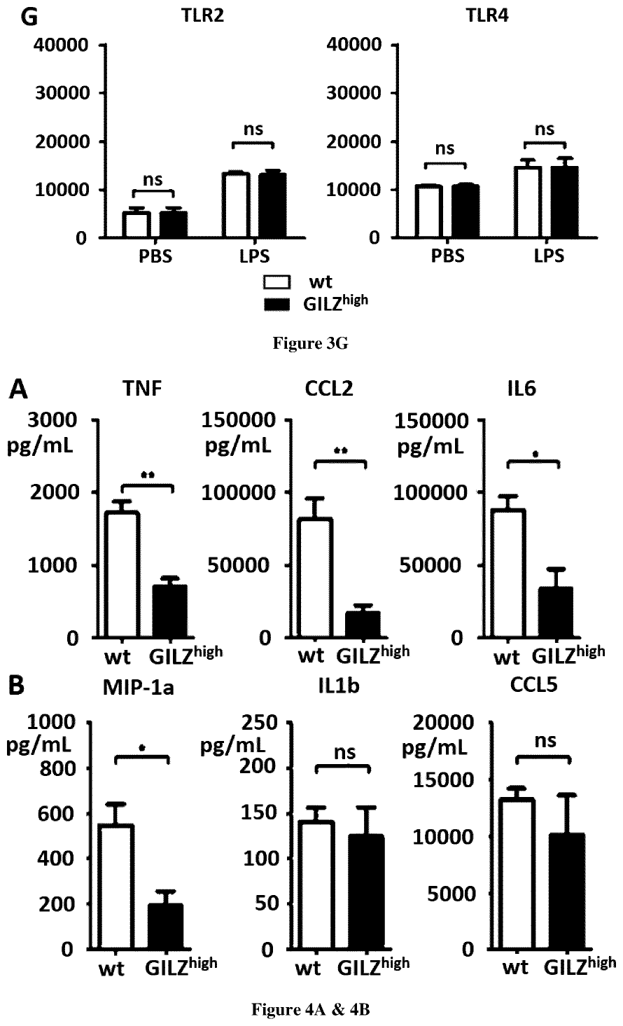

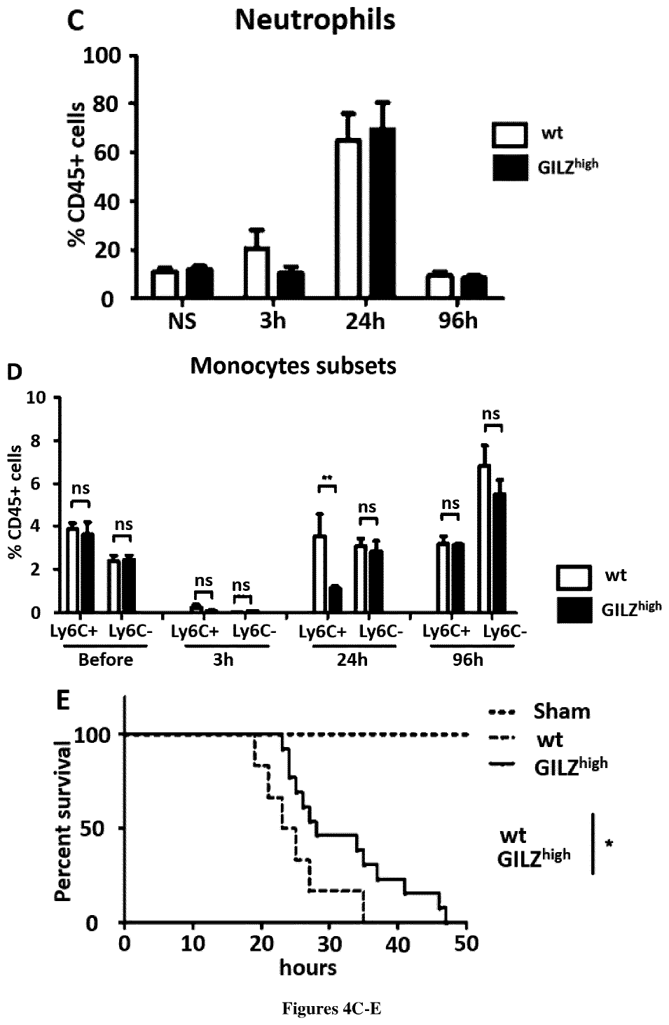

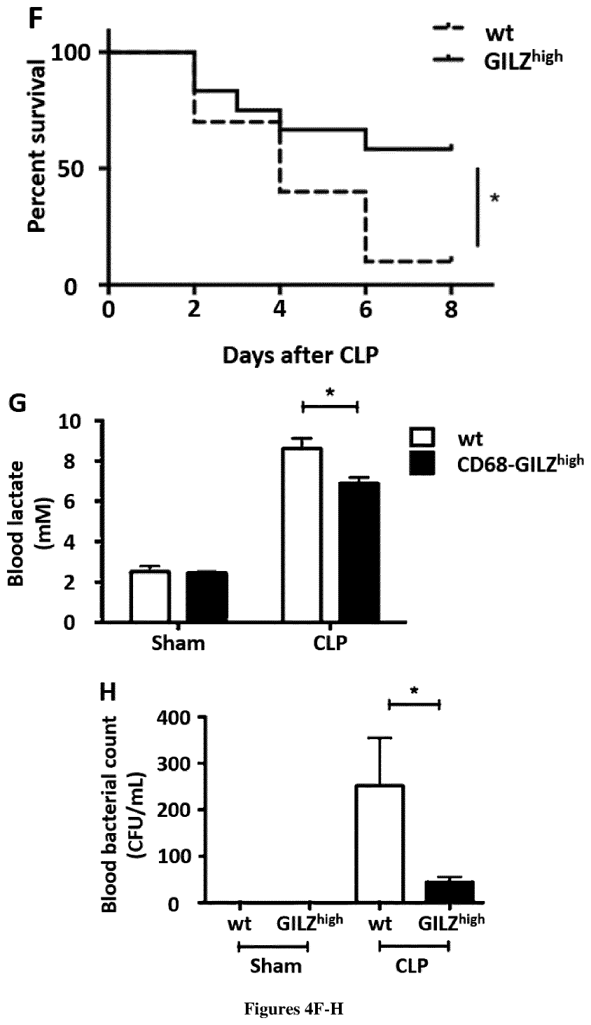

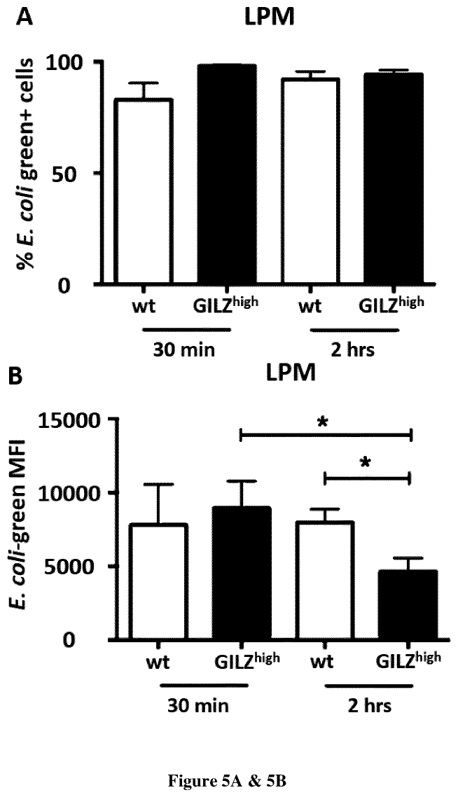

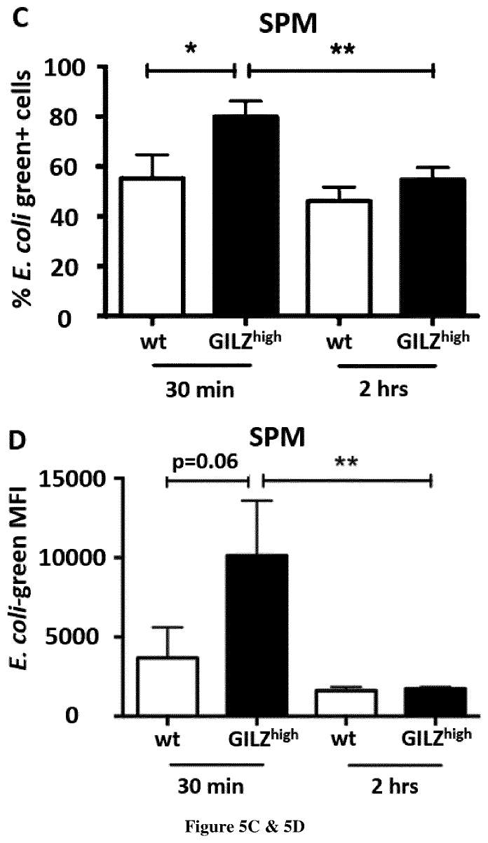

A recent study reported a downregulation of GILZ expression in total blood leukocytes of septic mice (18) while another one showed an up-regulation of GILZ expression in circulating neutrophils (9). This would indicate that GILZ expression is regulated in a cell-specific manner during sepsis. For our purposes, here, we evaluated the level of GILZ expression in peritoneal macrophages during LPS-induced endotoxemia. Large resident peritoneal macrophages (LPM, CD45⁺F4/80^{high}CD11b^{high}) and small peritoneal macrophages (SPM, CD45⁺F4/80^{int}CD11b^{int}) (FIG. 1A) were cell sorted from the peritoneal cavity of wild-type mice three hours after an i.p. injection of LPS (100 μg/mice) or PBS and tested for GILZ mRNA expression by qRT-PCR. The injection of LPS was associated with a significant change in peritoneal macrophage proportions. The frequency of LPM significantly decreased, while the percentage of SPM significantly increased compared to unstimulated mice (FIG. 1B) as described in a previous study (25). A significant reduction in GILZ mRNA level was observed in both LPM and SPM after in vivo LPS exposure while the level of TNF and IL6 mRNA was significantly increased (FIG. 1C).

To establish the direct effect of LPS on the regulation of GILZ expression, peritoneal macrophages were cultured ex vivo upon LPS exposure for up to eight hours. We thus focused on LPM that can respond to LPS in vivo and in vitro in contrast to SPM (25). In LPM, the levels of GILZ mRNA remained significantly low from two up to eight hours after in vitro LPS stimulation (FIG. 1D). Because the number of recovered LPM was insufficient to monitor the expression of GILZ by WB analysis, we repeated the experiment with BM-DM and confirmed in these settings a downregulation of GILZ expression both at the mRNA (FIG. 1E) and protein levels (Data not shown). This decrease of GILZ expression in LPM and BM-DM exposed in vitro to LPS was associated with a higher level of TNF and IL-6 mRNA by both populations of macrophages (FIGS. 1D and 1E).

We used the same experimental conditions to determine whether LPS exposure could also suppress GILZ expression in human monocytes prior to starting further investigations in patients with LPS-induced inflammatory disorders.

The decrease of GILZ expression was confirmed in CD14+ monocytes isolated from healthy donors at mRNA (FIG. 2A) and protein (Data not shown) levels with a linear correlation between gene expression and the protein (FIG. 2B). The suppression of GILZ expression in LPS-exposed human monocytes was associated with the induction of TNF secretion (FIG. 2A) as described in murine macrophages.

Next we monitored GILZ expression in monocytes purified from patients with septic shock as soon as diagnosis was confirmed. The systemic inflammatory response was documented by increased plasma CRP (204+/−58 mg/L), procalcitonin (7.7+/−2.4 ng/mL) and IL-6 levels (745.7 pg/mL+/−505.2 pg/mL). As repartition of monocyte subsets can be highly variable in septic patients (26) (27), we decided to isolate monocytes from PBMC using a magnetic kit optimized to preserve classical (CD45⁺CD14⁺CD16⁻), non-classical (CD45⁺CD14^{dim}CD16⁺) and intermediate (CD45⁺CD14⁺CD16⁺) monocytes. And as it had not previously been reported, we firstly looked at the level of GILZ mRNA in the three subsets of monocytes at steady-state. We showed that the level of GILZ was quite similar in classical, non-classical and intermediate monocytes coming from healthy donors.

The three monocyte subsets were equally represented in septic patients and healthy donors. A significantly lower expression of GILZ was found in monocytes from septic shock patients compared to healthy donors (FIG. 2C) emphasizing from a clinical perspective the need to more fully understand the contribution of GILZ in M/M responses during endotoxin-induced inflammation.

GILZ Expression Level Controls M/M Responses Exposed to LPS

We previously demonstrated that human monocytes transfected with a plasmid encoding GILZ secrete significantly less pro-inflammatory chemokines (Rantes, MIP-1α) after IFNγ exposition than mock-transfected cells (8). But at that time, we did not explore their secretion of TNF in response to LPS. To complete the phenotype of human GILZ-overexpressing monocytes, we transfected monocytes from healthy subjects using the same plasmids and procedure and assessed their TNF secretion after an exposure to LPS. We confirmed that human monocytes engineered to overexpress GILZ (FIG. 3A) produce significantly less TNF than their control counterparts (FIG. 3B).

In mice, evidence that the decrease of GILZ expression is a mandatory condition to elicit inflammatory responses in LPS-exposed M/M came from the transgenic mouse strain with an enforced expression of GILZ driven by the CD68 promoter (GILZ^{high}) leading to a targeted and permanent overexpression of GILZ in M/M. Their characteristics are described in our previous study (20). As expected, GILZ was overexpressed at mRNA and protein levels exclusively in their macrophages including peritoneal macrophages (FIG. 3C) and alveolar macrophages (FIG. 3D). We isolated LPM from GILZ^{high} and control mice and exposed them to LPS. LPM from GILZ^{high} mice retained a higher expression of GILZ after LPS stimulation compared to non-transgenic LPM (FIG. 3E), expressed significantly lower levels of TNF mRNA and significantly higher levels of IL10 mRNA (FIG. 3F).

The activation of M/M by bacteria requires the engagement of TLR4 for LPS and TLR2 for gram-positive bacteria. Our previous report showed that GILZ inhibits the expression of TLR-2 on human monocytic cells, which would partly explain why M/M with an overexpression of GILZ respond poorly to bacterial stimuli (8). We thus quantified by flow cytometry the expression of TLR4 as well as TLR2 on mouse LPM genetically modified to overexpress GILZ and their control counterparts. We showed that GILZ^{high}-LPM expressed similar level of TLR4 and TLR2 than genetically unmodified LPM (FIG. 3G). These results reinforced the hypothesis that GILZ inhibits the inflammatory responses of mouse M/M to LPS by controlling events downstream the triggering of TLR4 (28) and that the downregulation of GILZ is a pre-requisite for M/M to respond to LPS stimulation.

The Targeted Overexpression of GILZ in M/M Limits Systemic Inflammation and Enhances Lifetime in Murine Septic Shock

Macrophages contribute to the initiation of the systemic inflammatory response through the release of pro-inflammatory cytokines. Therefore, the anti-inflammatory cytokine profile of GILZ^{high}-LPM exposed in vitro to LPS prompted us to verify whether this could have an influence on the LPS-induced systemic inflammation. To address this question, we monitored the secretion of cytokines in the plasma of GILZ^{high} mice and control mice six hours after an i.p. injection of LPS after having verified that macrophages from GILZ^{high} mice kept a higher expression of GILZ upon in vivo LPS exposure. Significantly lower plasma levels of the pro-inflammatory cytokines and chemokines TNF, CCL2, IL-6 and MIP-1α (CCL3) were observed in GILZ^{high} transgenic mice (FIGS. 4A and 4B), suggesting attenuated systemic inflammatory response. The overexpression of GILZ in macrophages did not affect plasma levels of IL-1β or CCLS during endotoxemia (FIG. 4B).

Sepsis is also associated with an alteration of neutrophil and inflammatory monocyte counts in the blood (29) (30). We thus monitored changes in these populations in the blood of GILZ^{high} and non-transgenic control mice 3 hours, 24 hours and 96 hours after LPS-injection (FIGS. 4C and 4D). Neutrophil frequency was increased 24 hours post-injection in the same range in transgenic and non-transgenic mice (FIG. 4C). Twenty-four hours post-injection, a significant decrease in the frequency of inflammatory monocytes (Ly6C⁺) was observed in GILZ^{high} mice compared to non-transgenic mice (FIG. 4D).

We further evaluated whether the lower inflammatory response of GILZ^{high} mice exposed to LPS could improve clinical outcomes in septic shock. Septic shock is composed of an early inflammatory phase followed by a late immunosuppressive phase, which occurs due to the endotoxin tolerization of M/M. We chose two cecal ligation and puncture (CLP) procedures; a severe-grade to model the acute inflammatory phase of septic shock and a mild-grade, which recapitulates both early inflammatory and late immunosupressive phases. GILZ^{high} mice had a significantly increased lifetime compared to the control mice in both severe- and moderate-grade CLP models (FIGS. 4E and 4F). Additionally, the plasma lactate concentration and the bacteremia were quantified 18 hours after the mild-grade CLP. Elevated plasma levels of lactate are usually strongly associated with morbidity and mortality in sepsis. GILZ^{high} mice had significantly reduced lactate concentrations (FIG. 4G); a result that is consistent with the increased lifetime of these transgenic mice. GILZ^{high} mice had also significantly reduced bacterial counts in the blood compared to their littermate control mice (FIG. 41I).

Overall these results showed that the targeted overexpression of GILZ in the M/M is sufficient to control both the systemic inflammation and the bacterial spread during sepsis.

The Overexpression of GILZ Increases the Phagocytic Capacities of Macrophages

The reduced bacterial counts observed in the septic GILZ^{high} mice, prompted us to question the phagocytic capacities of M/M with an overexpression of GILZ. We measured in vivo the phagocytosis and bacterial clearance capacities of GILZ^{high}-macrophages using E. coli conjugated with pHrodo, a pH-sensitive green dye. The pHodro-fluorescent signal is emitted by the effect of phagosome acidification. The monitoring of the green fluorescence over time allows the quantification of macrophages with bacteria containing phagosomes and bacterial clearance, which results in a lost of the fluorescent signal. The former is assessed at early time points and the latter at late time points. GILZ^{high} transgenic and non-transgenic mice therefore received an i.p. injection of the pHrodo-conjugated E. coli. Peritoneal cells were harvested 30 min and 2 hours post-injection, stained to identify viable SPM and LPM and analyzed by flow cytometry.

Within thirty minutes, the frequency of pHrodro-green+ LPM reached 98%+/−1% in the GILZ^{high} mice and 83%+/−7.5% in the control littermate, with no statistical difference between both groups of mice (FIG. 5A). After two hours, values remained rather stable. These results indicate that the vast majority of LPM has ingested the pHrodro-conjugated E. coli in both GILZ^{high} and non-transgenic mice and in the same extent in both cases. The intensity of pHrodro-green signal was maximal at the early time point and similar between LPM from GILZ^{high} and control littermate while the signal significantly decreased in the GILZ^{high} mice after 2 hours (FIG. 5B). This result suggests that the bacterial clearance was faster in LPM with an overexpression of GILZ. As regards the SPM, a significant higher frequency of pHrodro-green+ cells was observed in GILZ^{high} mice at 30 min then the frequency significantly decreased after 2 hours (FIG. 5C). In line with this, the intensity of the pHrodro-green signal was higher at 30 min in SPM isolated from GILZ^{high} mice compared to those coming from control littermate (FIG. 5C). The data also showed at the late time point an almost 6-fold decrease in the green signal intensity in SPM from GILZ^{high} mice against a 2-fold drop in SPM from the control littermate (FIG. 5D). These experiments indicate that SPM with a high expression of GILZ have higher phagocytic capacities as well as a faster bacterial clearance.

Collectively, these results revealed increased phagocytic capacities of GILZ^{high} peritoneal macrophages. The GILZ-induced changes are dependent upon the type of peritoneal macrophages. GILZ improves the ingestion and destruction capacities of SPM while enhancing only the destructive abilities of LPM.

Discussion:

In the last decade, GILZ has been identified as a critical regulator of innate and adaptative immune responses (7, 31). To mention just a few examples, GILZ polarizes M/M into anti-inflammatory cells and dendritic cells into tolerant cells (8, 12, 13, 17, 28, 31, 32). Moreover a defective expression of GILZ has been related to chronic inflammatory diseases. The expression of GILZ is reduced in dendritic cells from patients with respiratory allergic diseases and absent in M/M located in the granuloma of patients with Crohn's disease (8) (13). In contrast, a high express of GILZ was reported in macrophages infiltrating Burkitt's tumors, to name just a few (8). So far, we do not know whether the defective expression of GILZ is the cause of immune diseases or a consequence of immune disorders. But, what we do know is that we can alter the outcome of immune diseases by modulating GILZ expression. A general approach of GILZ overexpression, i.e. in all cell types, has been tested by Ballegeer M. and coworkers and has increased the life-time of septic transgenic mice but with little effects on the systemic inflammation, an important aspect of the disease (18). Again, in the context of a general overexpression of GILZ, the administration of GILZ fusion protein can be protective by itself in an experimental model of encephalomyelitis (33). The complete opposite approach, which consists in the knockdown of GILZ has been used to increase the anti-tumoral immunity in a mouse model (34). Hence, GILZ has become a potential target in immunotherapies. In addition, many therapeutic actions of glucocorticoids, which are used in chronic inflammatory diseases and sepsis, according to recent advances in the field, are mediated by GILZ (7, 13, 15, 35). In the light of this, the GC-mediated metabolic abnormalities could also involved GILZ, which may offset the therapeutic benefits of GILZ. Indeed, GILZ is involved in GC-induced protein consumption in skeletal muscle cells (19) and its involvement in other metabolic abnormalities has not yet been explored. In this study, we wanted to explore the concept of a targeted modulation of GILZ expression in order to more accurately control the immune responses in sepsis without altering the metabolic pathways. The targeted population was here the M/M. M/M are key actors of host responses in sepsis. They recognize bacterial compounds mainly through TLR-4 for lipopolysaccharides (LPS) and TLR2 for gram-negative bacteria. They differentiate into M1-like polarized M/M and produce inflammatory cytokines including TNF mostly via the activation of the transcription factor NF-κB (36, 37).

We first reported that GILZ expression was decreased in monocytes purified from septic shock patients and was inhibited early in peritoneal macrophages from septic mice. This latter result contrasts with the increased expression of GILZ reported by Ballegeer et al. in peritoneal leukocytes isolated from septic mice (18). The leukocytes recruited in the peritoneal cavity during sepsis include a significant amount of neutrophils. An increase expression of GILZ has been reported in neutrophils during sepsis, which can explain the difference in GILZ expression between isolated peritoneal macrophages and total peritoneal leukocytes.

In in vitro assays, we showed that LPS suppresses GILZ within two hours in mouse LPM and human monocytes. These results reinforce previous observations demonstrating a down-regulation of GILZ in vitro in human and murine alveolar macrophages and BM-DM exposed to LPS.

To address the role of GILZ in M/M during the early inflammatory phase of sepsis, we used a severe-grade CLP procedure. Depending on the scientific requirements, the CLP model allows any kind of intensity modulation. In the severe-grade procedure, mice dye over a period of 48 hours (38). Attempting to demonstrate an effect is hard in this model as the inflammatory response is intense and death constant. However a benefit on mouse condition could only be related to GILZ modulation during the early inflammatory burst and not to the late multiple modifications in immunity including a role of GILZ in the ET (28). In this model, GILZ^{high} mice have a prolonged survival, indicating that GILZ's level of expression in M/M during the first phase of septic shock is a key factor on survival. In line with this, the CD68-GILZ^{high} transgenic mice show a significant reduction of proinflammatory cytokine and chemokine plasma levels, including TNF, IL-6 and CCL2 in sepsis settings. From a clinical and therapeutic stand point, this experimental data makes sense with clinical data from trials showing that a survival benefit was observed during earlier treatment of severe septic shock patients with corticosteroids, the most powerful inducers of GILZ expression in M/M (8). Furthermore, one of these studies reported that the beneficial effect of earlier corticosteroid treatment on patient survival was associated with a reduction of the proinflammatory responses of monocytes (39). In order to formally demonstrate that the beneficial effects of GC during sepsis require the up-regulation of GILZ expression in M/M, we should use myeloid-specific gilz knockout mice and show that these mice undergoing CLP are not rescued by a corticotherapy. But, for the time being, while the Cre/LoxP system typically used to target gene deletion to specific cell lineages is powerful, none of the available Cre driver line is M/M specific (40).

The second model of mild-grade CLP, during which mice dye over a period of seven days, indicates that the overexpression of GILZ maintained over time in M/M still improve mice outcome—despite the involvement of GILZ in the ET (28). During ET, M/M switch into anti-inflammatory cells, which express higher level of GILZ, possess the ability to release anti-inflammatory cytokines including IL-10 and contribute to resolution of inflammation (28). ET can also be seen as one of the components of the sepsis-induced long-term immune paralysis in which the risk of secondary infections is increased. GILZ could contribute to the resistance of mice experiencing a mild-grade CLP by regulating the early inflammatory responses on one hand and by improving the phagocytic capacities of M/M on the other hand. Indeed, the CD68-GILZ^{high} transgenic mice have a lower blood bacteremia after the CLP. Likewise, the overexpression of GILZ in peritoneal macrophages has significantly increased their ingestion and/or killing capacities depending on the subsets of peritoneal macrophages. We have already reported an impact of GILZ on the endocytosis pathways of dendritic cells, another phagocytic cell type (41). But in dendritic cells, the overexpression of GILZ limits the macropinocytosis through in part an inhibition of the p38 MAPK kinase pathway. Also GILZ does not influence the receptor-mediated phagocytosis of dendritic cells (41). In addition, it has been reported that GILZ overexpression has no impact on neutrophil phagocytosis (10). Overall, these results indicate that the GILZ-mediated effects on the endocytosis pathways vary according to the type of the phagocytic cell.

The effect of GILZ on macrophage response is associated with an inhibition of key transcription factors required for proinflammatory cytokine production, such as NF-κB (8). The mechanisms involved in the control of endocytosis pathways need to be clarified in M/M.

In summary, this study demonstrates a new role of GILZ in consequences of bacterial infections leading to septic shock showing that GILZ expression limited to monocytes and macrophages is sufficient to hamper the systemic inflammatory response in vivo while containing the bacterial spread. The sole GILZ overexpression in M/M creates an environment favorable to the fight against the bacterial infection while preserving the host against an excessive systemic inflammation. The cumulative result is a beneficial impact on the progression of the disease. Our data open a rationale for using drugs to modulate GILZ expression in earliest events of septic shock and the need to put in a lot of effort to identify cell specific inducers of GILZ. So far, we known that GC induce GILZ expression in immune and non-immune cells and that IL-4 and IL-13 are specific inducers of GILZ in M/M. Yet, it remains to identify other M/M specific inducers of GILZ that can be applied in clinical medicine and sepsis settings.

### REFERENCES

Throughout this application, various references describe the state of the art to which this invention pertains. The disclosures of these references are hereby incorporated by reference into the present disclosure.
1. Annane D, Bellissant E, Cavaillon J M. 2005. Septic shock. Lancet 365: 63-78
2. Angus D C, van der Poll T. 2013. Severe sepsis and septic shock. N Engl J Med 369: 2063
3. Annane D, Sharshar T. 2015. Cognitive decline after sepsis. Lancet Respir Med 3: 61-9
4. Dellinger R P, Levy M M, Rhodes A, Annane D, Gerlach H, Opal S M, Sevransky J E, Sprung C L, Douglas I S, Jaeschke R, Osborn T M, Nunnally M E, Townsend S R, Reinhart K, Kleinpell R M, Angus D C, Deutschman C S, Machado F R, Rubenfeld G D, Webb S A, Beale R J, Vincent J L, Moreno R, Surviving Sepsis Campaign Guidelines Committee including the Pediatric S. 2013. Surviving sepsis campaign: international guidelines for management of severe sepsis and septic shock: 2012. Crit Care Med 41: 580-637
5. Levy M M, Artigas A, Phillips G S, Rhodes A, Beale R, Osborn T, Vincent J L, Townsend S, Lemeshow S, Dellinger R P. 2012. Outcomes of the Surviving Sepsis Campaign in intensive care units in the USA and Europe: a prospective cohort study. Lancet Infect Dis 12: 919-24
6. Annane D, Renault A, Brun-Buisson C, Megarbane B, Quenot J P, Siami S, Cariou A, Forceville X, Schwebel C, Martin C, Timsit J F, Misset B, Ali Benali M, Colin G, Souweine B, Asehnoune K, Mercier E, Chimot L, Charpentier C, Francois B, Boulain T, Petitpas F, Constantin J M, Dhonneur G, Baudin F, Combes A, Bohe J, Loriferne J F, Amathieu R, Cook F, Slama M, Leroy O, Capellier G, Dargent A, Hissem T, Maxime V, Bellissant E, Network C-T. 2018. Hydrocortisone plus Fludrocortisone for Adults with Septic Shock. N Engl J Med 378: 809-18
7. Ayroldi E, Riccardi C. 2009. Glucocorticoid-induced leucine zipper (GILZ): a new important mediator of glucocorticoid action. FASEB J 23: 3649-58
8. Berrebi D, Bruscoli S, Cohen N, Foussat A, Migliorati G, Bouchet-Delbos L, Maillot M C, Portier A, Couderc J, Galanaud P, Peuchmaur M, Riccardi C, Emilie D. 2003. Synthesis of glucocorticoid-induced leucine zipper (GILZ) by macrophages: an anti-inflammatory and immunosuppressive mechanism shared by glucocorticoids and IL-10. Blood 101: 729-38
9. Espinasse M A, Hajage D, Montravers P, Piednoir P, Dufour G, Tubach F, Granger V, de Chaisemartin L, Noel B, Pallardy M, Chollet-Martin S, Biola-Vidamment A. 2016. Neutrophil expression of glucocorticoid-induced leucine zipper (GILZ) anti-inflammatory protein is associated with acute respiratory distress syndrome severity. Ann Intensive Care 6: 105
10. Espinasse M A, Pepin A, Virault-Rocroy P, Szely N, Chollet-Martin S, Pallardy M, Biola-Vidamment A. 2016. Glucocorticoid-Induced Leucine Zipper Is Expressed in Human Neutrophils and Promotes Apoptosis through Mc1-1 Down-Regulation. J Innate Immun 8: 81-96
11. Ayroldi E, Migliorati G, Bruscoli S, Marchetti C, Zollo O, Cannarile L, D'Adamio F, Riccardi C. 2001. Modulation of T-cell activation by the glucocorticoid-induced leucine zipper factor via inhibition of nuclear factor kappaB. Blood 98: 743-53
12. Hamdi H, Godot V, Maillot M C, Prejean M V, Cohen N, Krzysiek R, Lemoine F M, Zou W, Emilie D. 2007. Induction of antigen-specific regulatory T lymphocytes by human dendritic cells expressing the glucocorticoid-induced leucine zipper. Blood 110: 211-9
13. Karaki S, Garcia G, Tcherakian C, Capel F, Tran T, Pallardy M, Humbert M, Emilie D, Godot V. 2014. Enhanced glucocorticoid-induced leucine zipper in dendritic cells induces allergen-specific regulatory CD4(+) T-cells in respiratory allergies. Allergy 69: 624-31
14. Godot V, Garcia G, Capel F, Arock M, Durand-Gasselin I, Asselin-Labat M L, Emilie D, Humbert M. 2006. Dexamethasone and IL-10 stimulate glucocorticoid-induced leucine zipper synthesis by human mast cells. Allergy 61: 886-90
15. Cheng Q, Fan H, Ngo D, Beaulieu E, Leung P, Lo C Y, Burgess R, van der Zwan Y G, White S J, Khachigian L M, Hickey M J, Morand E F. 2013. GILZ overexpression inhibits endothelial cell adhesive function through regulation of NF-kappaB and MAPK activity. J Immunol 191: 424-33
16. Bereshchenko O, Coppo M, Bruscoli S, Biagioli M, Cimino M, Frammartino T, Sorcini D, Venanzi A, Di Sante M, Riccardi C. 2014. GILZ promotes production of peripherally induced Treg cells and mediates the crosstalk between glucocorticoids and TGF-beta signaling. Cell Rep 7: 464-75
17. Calmette J, Ellouze M, Tran T, Karaki S, Ronin E, Capel F, Pallardy M, Bachelerie F, Krzysiek R, Emilie D, Schlecht-Louf G, Godot V. 2014. Glucocorticoid-induced leucine zipper enhanced expression in dendritic cells is sufficient to drive regulatory T cells expansion in vivo. J Immunol 193: 5863-72
18. Ballegeer M, Vandewalle J, Eggermont M, Van Isterdael G, Dejager L, De Bus L, Decruyenaere J, Vandenbroucke R E, Libert C. 2018. Overexpression of Gilz Protects Mice Against Lethal Septic Peritonitis. Shock
19. Xiong J, Xu L, Qu W M, Li Z L, Shang Z H, Li Y H, Yang S H, Yang Z H. 2014. Roles of GILZ in protein metabolism of L6 muscle cells exposed to serum from septic rats. Genet Mol Res 13: 8209-19
20. Robert O, Boujedidi H, Bigorgne A, Ferrere G, Voican C S, Vettorazzi S, Tuckermann J P, Bouchet-Delbos L, Tran T, Hemon P, Puchois V, Dagher I, Douard R, Gaudin F, Gary-Gouy H, Capel F, Durand-Gasselin I, Prevot S, Rousset S, Naveau S, Godot V, Emilie D, Lombes M, Perlemuter G, Cassard A M. 2016. Decreased expression of the glucocorticoid receptor-GILZ pathway in Kupffer cells promotes liver inflammation in obese mice. J Hepatol 64: 916-24
21. Vincent J L, de Mendonca A, Cantraine F, Moreno R, Takala J, Suter P M, Sprung C L, Colardyn F, Blecher S. 1998. Use of the SOFA score to assess the incidence of organ dysfunction/failure in intensive care units: results of a multicenter, prospective study. Working group on “sepsis-related problems” of the European Society of Intensive Care Medicine. Crit Care Med 26: 1793-800
22. Ray A, Dittel B N. 2010. Isolation of mouse peritoneal cavity cells. J Vis Exp
23. Dandah A, Gautier G, Attout T, Fiore F, Lebourdais E, Msallam R, Daeron M, Monteiro R C, Benhamou M, Charles N, Davoust J, Blank U, Malissen B, Launay P. 2014. Mast cells aggravate sepsis by inhibiting peritoneal macrophage phagocytosis. J Clin Invest 124: 4577-89
24. Toscano M G, Ganea D, Gamero A M. 2011. Cecal ligation puncture procedure. J Vis Exp
25. Ghosn E E, Cassado A A, Govoni G R, Fukuhara T, Yang Y, Monack D M, Bortoluci K R, Almeida S R, Herzenberg L A, Herzenberg L A. 2010. Two physically, functionally, and developmentally distinct peritoneal macrophage subsets. Proc Natl Acad Sci USA 107: 2568-73
26. Nockher W A, Scherberich J E. 1998. Expanded CD14+CD16+ monocyte subpopulation in patients with acute and chronic infections undergoing hemodialysis. Infect Immun 66: 2782-90
27. Fingerle G, Pforte A, Passlick B, Blumenstein M, Strobel M, Ziegler-Heitbrock H W. 1993. The novel subset of CD14+/CD16+ blood monocytes is expanded in sepsis patients. Blood 82: 3170-6
28. Hoppstadter J, Kessler S M, Bruscoli S, Huwer H, Riccardi C, Kiemer A K. 2015. Glucocorticoid-induced leucine zipper: a critical factor in macrophage endotoxin tolerance. J Immunol 194: 6057-67
29. Chignard M, Balloy V. 2000. Neutrophil recruitment and increased permeability during acute lung injury induced by lipopolysaccharide. Am J Physiol Lung Cell Mol Physiol 279: L1083-90
30. O'Connell P A, Surette A P, Liwski R S, Svenningsson P, Waisman D M. 2010. S100A10 regulates plasminogen-dependent macrophage invasion. Blood 116: 1136-46
31. Pepin A, Biola-Vidamment A, Latre de Late P, Espinasse M A, Godot V, Pallardy M. 2015. [TSC-22D proteins: new regulators of cell homeostasis?]. Med Sci (Paris) 31: 75-83
32. Cohen N, Mouly E, Hamdi H, Maillot M C, Pallardy M, Godot V, Capel F, Balian A, Naveau S, Galanaud P, Lemoine F M, Emilie D. 2006. GILZ expression in human dendritic cells redirects their maturation and prevents antigen-specific T lymphocyte response. Blood 107: 2037-44
33. Srinivasan M, Janardhanam S. 2011. Novel p65 binding glucocorticoid-induced leucine zipper peptide suppresses experimental autoimmune encephalomyelitis. J Biol Chem 286: 44799-810
34. Lebson L, Wang T, Jiang Q, Whartenby K A. 2011. Induction of the glucocorticoid-induced leucine zipper gene limits the efficacy of dendritic cell vaccines. Cancer Gene Ther 18: 563-70
35. Yang N, Zhang W, Shi X M. 2008. Glucocorticoid-induced leucine zipper (GILZ) mediates glucocorticoid action and inhibits inflammatory cytokine-induced COX-2 expression. J Cell Biochem 103: 1760-71
36. Hotchkiss R S, Monneret G, Payen D. 2013. Sepsis-induced immunosuppression: from cellular dysfunctions to immunotherapy. Nat Rev Immunol 13: 862-74
37. Stearns-Kurosawa D J, Osuchowski M F, Valentine C, Kurosawa S, Remick D G. 2011. The pathogenesis of sepsis. Annu Rev Pathol 6: 19-48
38. Rittirsch D, Huber-Lang M S, Flierl M A, Ward P A. 2009. Immunodesign of experimental sepsis by cecal ligation and puncture. Nat Protoc 4: 31-6
39. Katsenos C S, Antonopoulou A N, Apostolidou E N, Ioakeimidou A, Kalpakou G T, Papanikolaou M N, Pistiki A C, Mpalla M C, Paraschos M D, Patrani M A, Pratikaki M E, Retsas T A, Savva A A, Vassiliagkou S D, Lekkou A A, Dimopoulou I, Routsi C, Mandragos K E, Hellenic Sepsis Study G. 2014. Early administration of hydrocortisone replacement after the advent of septic shock: impact on survival and immune response*. Crit Care Med 42: 1651-7
40. McCubbrey A L, Allison K C, Lee-Sherick A B, Jakubzick C V, Janssen W J. 2017. Promoter Specificity and Efficacy in Conditional and Inducible Transgenic Targeting of Lung Macrophages. Front Immunol 8: 1618
41. Calmette J, Bertrand M, Vetillard M, Ellouze M, Flint S, Nicolas V, Biola-Vidamment A, Pallardy M, Morand E, Bachelerie F, Godot V, Schlecht-Louf G. 2016. Glucocorticoid-Induced Leucine Zipper Protein Controls Macropinocytosis in Dendritic Cells. J Immunol 197: 4247-56

1. A method of predicting the survival time of a patient suffering from sepsis comprising the steps of:
i) providing a macrophage or monocyte sample from the patient,
ii) determining the expression level of GILZ in said macrophage or monocyte sample, and
iii) comparing the expression level determined at step ii) with a predetermined reference level
wherein detecting differences between the expression level determined at step ii) and the predetermined reference value indicates that the patient will have a short or long survival time.

2. A method of determining whether a patient suffering from sepsis is eligible for treatment with a corticoid and treating the patient with the corticoid, comprising the steps of:
i) providing a sample comprising macrophages and/or monocytes from the patient,
ii) culturing the macrophages and/or monocytes in vitro both in the presence and in the absence of the corticosteroid,
iii) determining the expression level of GILZ in the macrophages and/or monocytes cultured in the presence of the corticosteroid and the macrophages and/or monocytes cultured in the absence of the corticosteroid
iv) administering a therapeutically effective amount of the corticosteroid to the patient when a ratio between the expression
level of the macrophages and/or monocytes cultured in the presence of the corticosteroid to the macrophages and/or monocytes cultured in the absence of the corticosteroid is greater than 1.

3. The method of claim 2 wherein the patient suffers from systemic inflammatory response syndrome.

4. The method of claim 2 wherein the patient suffers from acute respiratory distress syndrome.

5. The method of claim 2 wherein the corticoid is selected from the group consisting of hydrocortisone (Cortisol), cortisone acetate, prednisone, prednisolone, methylprednisolone, deflazacort, betamethasone, triamcinolone, beclometasone, Paramethasone, fluticasone, fludrocortisone acetate, deoxycorticosterone acetate (DOCA), Fluprednisolone, fluticasone propionate, budesonide, beclomethasone dipropionate, flunisolide and triamcinolone acetonide.

6. The method of claim 2 wherein the corticosteroid is dexamethasone.

7. The method of claim 2 wherein the sample is a sample of blood monocytes.

8. The method of claim 2 wherein the sample is sample of alveolar macrophages.

9. The method of claim 2 wherein the expression level of GILZ is determined by PCR or flow cytometry.

10-12. (canceled)


SEQUENCE LISTING (1 sequence)

SEQ ID NO: 1 - protein, 134 aa, Homo sapiens
MNTEMYQTPMEVAVYQLHNFSISFFSSLLGGDVVSVKLDNSASGASVVAIDNKIEQAMDLVKNHLMYAVREEVEILKEQIRELVEKNSQLERENTLLKTLASPEQLEKFQSCLSPEEPAPESPQVPEAPGGSAV